(19) United States
(12) Patent Application Publication
(10) Pub. No.: US 2022/0195379 A1
(43) Pub. Date: Jun. 23, 2022

(54) METHOD OF TESTTING INHIBITORY EFFECT OF CARBON DIOXIDE ON SPOILAGE CAUSING ABILITY OF SHEWANELLA PUTREFACIENS

(71) Applicant: Shanghai Ocean University, Shanghai (CN)

(72) Inventors: Jing XIE, Shanghai (CN); Peiyun LI, Shanghai (CN); Jinfeng WANG, Shanghai (CN); Yueming CHEN, Shanghai (CN)

(21) Appl. No.: 17/693,309

(22) Filed: Mar. 11, 2022

(30) Foreign Application Priority Data
Jan. 19, 2022 (CN) 202210058233.1

(51) Int. Cl. C12N 1/04; C12N 1/20

(52) U.S. Cl. CPC C12N 1/04; C12N 2500/02; C12N 1/205

(57) ABSTRACT

Disclosed is a method of testing an inhibitory effect of carbon dioxide on a spoilage causing ability of Shewanella putrefaciens, which includes activation, preparation of a bacterial suspension, preparation and treatment of samples, inoculation, transfer to packaging bag, modified atmosphere packaging, refrigeration and performance detection. After stored for 0-18 days, three inoculated samples are randomly selected and tested to evaluate inhibitory effects of different CO₂ concentrations on the spoilage causing ability of Shewanella putrefaciens.

### CROSS-REFERENCE TO RELATED APPLICATIONS

This application claims the benefit of priority from Chinese Patent Application No. 202210058233.1, filed on Jan. 19, 2022. The content of the aforementioned application, including any intervening amendments thereto, is incorporated herein by reference.

### TECHNICAL FIELD

This application relates to food preservation, and more particularly to a method of testing an inhibitory effect of carbon dioxide on a spoilage causing ability of Shewanella putrefaciens.

### BACKGROUND

Shewanella putrefaciens is the predominant specific spoilage organism (SSO) for fresh aquatic products (such as fishes, shrimps, and shells). During the cold storage of the aquatic products, the Shewanella putrefaciens is capable of reducing trimethylamine oxide into H₂S gas, resulting in the deterioration of the aquatic products. At the same time, the extracellular protease secreted by the Shewanella putrefaciens can contribute to the protein degradation, causing the deterioration of nutritional quality and flavor quality of the aquatic products. Moreover, the growth and proliferation of the Shewanella putrefaciens can produce a biofilm, which involves a certain resistance to dry condition and antibiotics, and is extremely difficult to be completely removed, continuously causing the deterioration. Biofilms attaching to the food processing equipment will cause energy loss and decline of the heat transfer efficiency, reducing the production efficiency, as well as causing corrosion of the food processing equipment. In the storage process of the aquatic products, the biofilms formed by the proliferation of the Shewanella putrefaciens will exacerbate the spoilage of the aquatic products, thus seriously threatening the processing and storage of the aquatic products. Hence, the inhibition of growth and spoilage potential of the Shewanella putrefaciens is essential for improving the processing and storage of the aquatic products.

### SUMMARY

An object of this disclosure is to provide a method of testing an inhibitory effect of carbon dioxide on a spoilage causing ability of Shewanella putrefaciens, in which the Shewanella putrefaciens is inoculated to a sterilized large yellow croaker fillet, and stored under different concentrations of carbon dioxide (CO₂); and through observing the growth change of the Shewanella putrefaciens and the quality change of the sterilized large yellow croaker fillet, and detecting the growth and proliferation rate of the Shewanella putrefaciens under different storage conditions, the inhibitory effect of CO₂ on the spoilage causing ability of Shewanella putrefaciens can be evaluated. This application provides a reference for the effective reduction of the growth and proliferation rate of Shewanella putrefaciens and its spoilage causing ability.

Technical solutions of this application are specifically described as follows. The disclosure provides of testing an inhibitory effect of carbon dioxide on a spoilage causing ability of Shewanella putrefaciens, comprising:

(S1) thawing frozen Shewanella putrefaciens followed by inoculation to a trypticase soy broth (TSB); and culturing the Shewanella putrefaciens under shaking for 10-14 h to obtain a primary culture solution;

(S2) inoculating the primary culture solution into a liquid medium followed by culture under shaking for 4-8 h to obtain a bacterial suspension with a concentration of 10⁶-10⁸ CFU/mL;

(S3) cutting an aquatic product material into a plurality of slices each with a mass of 80-120 g followed by washing with 0-4° C. water and drying to prepare a plurality of samples;

(S4) immersing the plurality of samples in an ethanol solution for 20-40 s followed by washing with sterile distilled water 2-4 times and ultraviolet sterilization for 15-25 min to obtain a plurality of sterilized samples; diluting the bacterial suspension to a concentration of 10³-10⁵ CFU/mL; and inoculating a diluted bacterial suspension to the plurality of sterilized samples to obtain a plurality of inoculated samples;

(S5) transferring the plurality of inoculated samples respectively to a plurality of modified atmosphere packaging bags;

(S6) turning on a modified atmosphere packaging machine and a vacuum pump to package the plurality of inoculated samples; wherein protective gases in the plurality of modified atmosphere packaging bags are each composed of 0-100% by volume of CO₂ and N₂, and vary in CO₂ content;

(S7) after packaging, storing the plurality of inoculated samples in a refrigerator at 4.0±0.5° C.; and

(S8) after stored for 0-18 days, for each protective gas, randomly selecting three from the plurality of inoculated samples as parallel groups for test to evaluate inhibitory effects of different concentrations of CO₂ on the spoilage causing ability of Shewanella putrefaciens.

In some embodiments, in step (S1), the frozen Shewanella putrefaciens is stored lower than or equal to −80° C.

In some embodiments, in step (S1), the frozen Shewanella putrefaciens is thawed at 30±2° C.

In some embodiments, in step (S1), an amount of the TSB medium is 5-10 mL.

In some embodiments, in step (S2), an inoculation amount of the primary culture solution is 0.5-1.5% by mass.

In some embodiments, the liquid medium in step (S2) is the TSB medium.

In some embodiments, the aquatic product in step (S3) is a large yellow croaker with a weight of 1000±50 g; and the large yellow croaker is oxygenated in a foam box during transportation.

In some embodiments, in step (S3), the aquatic product is processed by bloodletting, internal organ removal, and head cutting.

In some embodiments, in step (S4), a mass concentration of the ethanol solution is 70-80%; and a mass ratio of the ethanol solution to the plurality of samples is (2-4):1.

In some embodiments, in step (S4), the diluted bacterial suspension is 5-15% by weigh of each of the plurality of sterilized samples such that a bacterial concentration of each of the plurality of sterilized samples is lower than or equal to 10⁵ CFU/mL.

In some embodiments, in step (S5), the plurality of modified atmosphere packaging bags are polyvinylidene chloride packaging bags.

In some embodiments, in step (S6), a ratio of a volume the protective gas in each of the plurality of modified atmosphere packaging bags to a weight of each of the plurality of inoculated samples is 2-4 (mL):1 (g); a vacuuming time is set to 5-15 s; a filling time of the protective gas is 3-5 s; a temperature for heat sealing is 125-150° C.; a protective gas source pressure is 4-6 kg/cm²; a power gas source pressure is 7-8 kg/cm²; and after the modified atmosphere packaging machine is operated, a mouth of each of the plurality of modified atmosphere packaging bags containing an inoculated sample is placed at a position where gas displacement and heat sealing are performed for modified atmosphere packaging.

In some embodiments, in step (S8), the parallel groups are tested for colony number, biofilm growth, adenosine triphosphate content, thiol content, tertiary structure of myofibrillar protein, ultrastructure of the myofibrillar protein and hardness. The colony number is detected through the following steps.

(S1) 5.0 g of a sample is mixed uniformly in 45 mL of a sterilized saline solution to obtain a sample dispersion.

(S2) 1 mL of the sample dispersion is pipetted using a 1 mL sterile pipette and injected into 9 mL of the sterilized saline solution along the tube wall for a 10-fold dilution to obtain a primary sample dilution.

(S3) The primary sample dilution is diluted according to step (S2) to obtain a secondary sample dilution, and so on until a denary sample dilution was obtained. (S4) Three sample dilutions are randomly selected from the above ten dilutions. Subsequently, 1 mL of each selected sample dilution is added to individual sterilized petri dishes, and meanwhile, two sterilized petri dishes are respectively added with 1 mL of the sterilized saline solution, and kept open in a clean bench for blank control.

(5) The above petri dishes are respectively added with 15-20 mL of a plate count agar medium pre-cooled to 45±0.5° C., and rotated for uniform mix. After being solidified, the plates are invertedly cultured in a biochemical incubator at 30±1° C. for 72±3 h.

(6) The total number of colonies in the petri dishes is counted by the plate counting method, and the sterilized saline solution is used as a blank control. The petri dishes with 30-300 colonies are selected for counting.

The biofilm growth is detected through the following steps.

A 48-well plate is added with 1 mL of a sample, and subjected to standing culture at 4.0±0.5° C. for 24 h. After that, the supernatant is removed, and the residue is washed twice with a 0.01 M sterilized phosphate buffer (pH 7.0) to remove the free bacterial cells, dried at 50±1° C. for 30 min, and then stained with a 0.2% crystal violet solution for 15 min. The stained product is washed with water to remove the free crystal violet, dried at 50±1° C. for 30 min, and then immersed in a 95% ethanol solution for 5 min to allow the crystalline violet attached to the biofilm to be dissolved in the ethanol solution. At last, the absorbance of the ethanol solution containing the crystalline violet is measured under 600 nm, and the biofilm growth is characterized by the change of absorbance.

The adenosine triphosphate content is determined via an adenosine triphosphate kit.

The thiol content is determined through the following steps.

2 g of a sample is evenly mixed with 20 mL of a cooled Tris-buffer A (pH 7.0) containing 0.05 M KC1 and 20 mM Tris-maleate, and centrifuged at 4° C. and 10,000×g for 15 min. The supernatant is discarded, and the residue is washed again through the above-described steps to obtain a precipitate. Subsequently, the precipitate is mixed with 20 mL of a cooled Tris-buffer B (pH 7.0) containing 0.6 M KC1 and 20 mM Tris-maleate, kept at 4° C. for 3 h, and centrifuged at 10000×g for 15 min to obtain a supernatant as a myofibrillar protein extract solution. Finally, the total thiol content of the myofibrillar protein extract solution is determined via a total sulfhydryl group content assay kit.

The myofibrillar protein tertiary structure is observed as follows. The myofibrillar protein extract solution is freeze-dried, and scanned at an excitation wavelength of 295 nm and an emission wavelength of 300-410 nm via an emission scanning mode of a fluorescence spectrophotometer to determine the intrinsic fluorescence intensity (IFI) to characterize the tertiary structure of the myofibrillar protein.

The myofibrillar protein ultrastructure is characterized as follows.

A sample is cut into a plurality of blocks with a size of 3mm×3mm×1.5mm, which are added with a 2.5% glutaraldehyde solution and kept at 4° C. for 24 h for fixation. After that, the supernatant is removed, and the residue is rinsed with a 0.1 M phosphate buffer solution (pH 7.3) three times each for 15 min. The rinsed samples are subjected to gradient elution sequentially with 30%, 50%, 70%, 80%, 90%, 95% and 100% ethanol solutions, washed with isoamyl acetate, freeze-dried, sprayed with gold in a sputter coater for 1 min, and observed under a scanning electron microscope at an accelerating voltage of 20 kV. The samples obtained after the isoamyl acetate washing are cut into several secondary samples with a size of 1 mm×1 mm×1 mm, fixed with a 2.5% glutaraldehyde solution for 10 min, eluted successively with the phosphate buffer solution and 70%, 80%, 90%, 95% and 100% ethanol solutions each for 10 min, embedded in epoxy resin, and observed by a transmission electron microscope.

The hardness is determined through the following steps.

The sample is cut into a plurality of blocks with a size of 15 mm×15 mm×15 mm, and detected under a texture profile analysis (TPA) mode, where the descending speed of the probe (p/5) before the detection is 2.00 mm/s; the detection speed is 1.00 mm/s; the returning speed of the probe after the detection is 5.00 mm/s; the compression ratio is 40%; the trigger force is 5.0 g; and the data acquisition rate is 200.00 points/s. Three parallel experiments are conducted for each group, and the results are averaged.

In the method provided herein, the cooled plate count agar medium is poured into a petri dish, and the petri dish is rotated to allow uniform mix. After being solidified, the plate is inverted in a biochemical incubator for culture, and the total number of colonies is determined by the plate counting method with the sterilized saline solution as a blank control. All samples of the present disclosure are stored at 4±0.5° C., and the inoculated samples are packaged in a protective gas containing 0-100% by volume of CO₂. It has been proved that CO₂ can effectively reduce the growth and propagation rate of the Shewanella putrefaciens and weaken the spoilage and degradation capacity for the inoculated samples, slowing down the deterioration rate of the quality of aquatic products. At the same time, CO₂ is safe, environmental, and low-cost, so the method provided herein has a promising commercial application value for the inhibition of the spoilage-causing performance of the Shewanella putrefaciens.

### BRIEF DESCRIPTION OF THE DRAWINGS

FIG. 1 is a curve diagram illustrating a change of the total number of colonies over storage time;

FIG. 2 is a curve diagram illustrating a change of biofilm over storage time;

FIG. 3 is a curve diagram illustrating a change of carbonyl concentration over storage time;

FIG. 4 is a curve diagram presenting a change of an activity of Ca²⁺-ATPase over storage time;

FIG. 5 shows detection results of a tertiary structure of myofibrillar protein;

FIG. 6A is a scanning electron microscope (SEM) image of an ultrastructure of the myofibrillar protein, where the upper picture: untreated sample; and the lower five pictures: treated samples after stored under different protective gases for 18 days;

FIG. 6B is a transmission electron microscope (TEM) image of the ultrastructure of the myofibrillar protein, where the upper picture: untreated sample; and the lower five pictures: treated samples after stored under different protective gases for 18 days; and

FIG. 7 is a curve diagram showing a change of hardness value over storage time.

In the drawings, AP is a control group packaged in the air; MAP1 is a test group packaged in 100% N₂; MAP2 is a test group packaged in a protective gas mix of 20% by volume of CO₂ and 80% by volume of N₂; MAP3 is a test group packaged in a protective gas mix of 60% by volume of CO₂ and 40% by volume of N₂; and MAP4 is a test group packaged in 100% CO₂.

### DETAILED DESCRIPTION OF EMBODIMENTS

The modified atmosphere packaging bags used herein have a size of 28 cm×28 cm.

After stored respectively in protective gases varying in CO₂ concentration for 18 days, the samples were analyzed for the colony number, biofilm growth, adenosine triphosphate content, thiol content, tertiary structure of myofibrillar protein, ultrastructure of the myofibrillar protein and hardness, and the results are shown in Table 1.

TABLE 1
Effects of CO₂ concentration on spoilage
causing ability of the Shewanella putrefaciens

| Protective gas | MAP1 | MAP2 | MAP3 | MAP4 | AP |
|---|---|---|---|---|---|
| Total number of colonies | 7.37 | 6.39 | 6.02 | 10.26 | 7.79 |
| (lg CFU/g) | | | | | |
| Biofilm (absorbance) | 1.63 | 1.45 | 0.42 | 0.23 | 2.17 |
| Adenosine triphosphate | 63.93 | 54.05 | 47.43 | 44.11 | 56.02 |
| content (nM) | | | | | |
| Thiol content (μmol/g pro) | 14.89 | 13.17 | 12.32 | 32.82 | 17.01 |
| Tertiary structure (A.U.) | 0.94 | 1.10 | 1.23 | 0.58 | 0.92 |
| Hardness | 5.02 | 5.36 | 5.65 | 2.07 | 4.25 |

Notes:
AP: air;
MAP1: 100% N₂;
MAP2: 20% CO₂ + 80% N₂;
MAP3: 60% CO₂ + 40% N₂; and
MAP4: 100% CO₂.

To facilitate the understanding and implementation, the disclosure will be described in detail below with reference to the embodiments and drawings.

### EXAMPLE 1

Pre-frozen Shewanella putrefaciens (stored at −80° C. or less) was thawed at 30±2° C., inoculated to 5-10 mL of TSB, and cultured under shaking for 12 h to obtain a primary culture solution.

The primary culture solution was inoculated into another TSB liquid medium at an inoculation amount of 1% by weight, which was cultured under shaking for 6 h to obtain a bacterial suspension with a concentration of 10⁷ CFU/mL.

A large yellow croaker with a weight of 1000±50 g was processed, and cut into a plurality of 100-g slices, which were washed with 0-4° C. water, and dried to prepare a plurality of samples. The large yellow croaker was oxygenated in a foam box during the transportation, and processed by bloodletting, evisceration, and head removal.

The samples were immersed into a 75% ethanol solution for 30 s, washed with sterile distilled water 3 times, and subjected to ultraviolet sterilization for 20 min to obtain a plurality of sterilized samples, where a mass ratio of the ethanol solution to the samples was 3:1. The bacterial suspension was diluted to a concentration of 10⁴ CFU/mL, and inoculated to the sterilized samples to obtain a plurality inoculated samples, where the diluted bacterial suspension was 10% by weight of each sterilized sample such that a bacterial concentration of each sterilized sample was lower than or equal to 10⁵ CFU/mL.

The inoculated samples were respectively transferred to a plurality of modified atmosphere packaging bags made from polyvinylidene chloride.

A modified atmosphere packaging machine and a vacuum pump were turned on to package the inoculated samples, where the protective gases were respectively set to (by volume): 100% N₂ (MAP1), 20% CO₂₊₈₀% N₂ (MAP2), 60% CO₂₊₄₀% N₂ (MAP3) and 100% CO₂ (MAP4). During the packaging process, a ratio of a volume of the protective gas to a weight of the sample was 3(mL):1(g), other parameters were set as follows: vacuuming time: 10 s; filling time of the protective gas: 4 s; heat sealing temperature: 140° C.; protective gas source pressure: 5 kg/cm²; and power gas source pressure: 7.5 kg/cm². After the modified atmosphere packaging machine was operated stably, a mouth of a modified atmosphere packaging bag containing an inoculated sample was placed at a position where the gas displacement and heat sealing were performed for modified atmosphere packaging.

After packaging, the plurality inoculated samples were stored in a refrigerator at 4.0±5.0° C.

For each protective gas composition, three inoculated samples were randomly selected respectively after stored for 0, 3, 6, 9, 12, 15 and 18 days, and tested to evaluate the inhibitory effects of different CO₂ concentrations on the spoilage causing ability.

The test items included colony number, biofilm growth, adenosine triphosphate content, thiol content, tertiary structure of myofibrillar protein, myofibrillar protein ultrastructure and hardness.

The colony number was detected through the following steps.

(S1) 5.0 g of a sample was mixed uniformly in 45 mL of a sterilized saline solution to obtain a sample dispersion.

(S2) 1 mL of the sample dispersion was pipetted using a 1 mL sterile pipette and injected into 9 mL of the sterilized saline solution along the tube wall for a 10-fold dilution to obtain a primary sample dilution.

(S3) The primary sample dilution was diluted according to step (S2) to obtain a secondary sample dilution, and so on until a denary sample dilution was obtained.

(S4) Three sample dilutions were randomly selected from the above ten dilutions. Subsequently, 1 mL of each selected sample was pipetted to a sterilized petri dish, and meanwhile, two sterilized petri dishes were respectively added with 1 mL of the sterilized saline solution, and kept open in a clean bench for blank control.

(5) The above petri dishes were respectively added with 15-20 mL of a plate count agar medium pre-cooled to 45±0.5° C., and rotated for uniform mix. After being solidified, the plates were invertedly cultured in a biochemical incubator at 30±1° C. for 72±3 h.

(6) The total number of colonies in the petri dishes was counted by the plate counting method, and the sterilized saline solution was used as a blank control. The petri dishes with 30-300 colonies were selected for counting.

The biofilm growth was detected through the following steps.

A 48-well plate was added with 1 mL of a sample, and subjected to standing at 4.0±0.5° C. for 24 h. After that, the supernatant was removed, and the residue was washed twice with a 0.01 M sterilized phosphate buffer (pH 7.0) to remove the free bacterial cells, dried at 50±1° C. for 30 min, and then stained with a 0.2% crystal violet solution for 15 min. The stained product was washed with water to remove the free crystal violet, dried at 50±1° C. for 30 min, and then immersed in a 95% ethanol solution for 5 min to allow the crystalline violet attached to the biofilm to be dissolved in the ethanol solution. At last, the absorbance of the ethanol solution containing the crystalline violet was measured under 600 nm, and the biofilm growth was characterized by the change of absorbance.

The adenosine triphosphate content was determined via an adenosine triphosphate kit.

The thiol content was determined through the following steps.

2 g of a sample was evenly mixed with 20 mL of a cooled Tris-buffer A (pH 7.0) containing 0.05 M KC1 and 20 mM Tris-maleate, and centrifuged at 4° C. and 10,000×g for 15 min. The supernatant was discarded, and the residue was washed again through the above-described steps to obtain a precipitate. Subsequently, the precipitate was mixed with 20 mL of a cooled Tris-buffer B (pH 7.0) containing 0.6 M KC1 and 20 mM Tris-maleate, kept at 4° C. for 3 h, and centrifuged at 10000 x g for 15 min to obtain a supernatant as a myofibrillar protein extract solution. Finally, the total thiol content of the myofibrillar protein extract solution was determined via a total sulfhydryl group content assay kit.

The myofibrillar protein tertiary structure was observed as follows.

The myofibrillar protein extract solution was freeze-dried, and scanned at an excitation wavelength of 295 nm and an emission wavelength of 300-410 nm via an emission scanning mode of a fluorescence spectrophotometer to determine the intrinsic fluorescence intensity (IFI) to characterize the tertiary structure of the myofibrillar protein.

The myofibrillar protein ultrastructure was characterized as follows.

A sample was cut into a plurality of blocks with a size of 3 mm×3 mm×1.5 mm, which were added with a 2.5% glutaraldehyde solution and kept at 4° C. for 24 h for fixation. After that, the supernatant was removed, and the residue was rinsed with a 0.1 M phosphate buffer solution (pH 7.3) three times each for 15 min. The rinsed samples were subjected to gradient elution sequentially with 30%, 50%, 70%, 80%, 90%, 95% and 100% ethanol solutions, washed with isoamyl acetate, freeze-dried, sprayed with gold in a sputter coater for 1 min, and observed under a scanning electron microscope at an accelerating voltage of 20 kV. The samples obtained after the isoamyl acetate washing were cut into several secondary samples with a size of 1 mm×1 mm×1 mm, fixed with a 2.5% glutaraldehyde solution for 10 min, eluted successively with the phosphate buffer solution and 70%, 80%, 90%, 95% and 100% ethanol solutions each for 10 min, embedded in epoxy resin, and observed by a transmission electron microscope.

The hardness was determined through the following steps.

The sample was cut into a plurality of blocks with a size of 15 mm×15 mm×15 mm, and detected under a texture profile analysis (TPA) mode, where the descending speed of the probe (p/5) before the detection was 2.00 mm/s; the detection speed was 1.00 mm/s; the returning speed of the probe after the detection was 5.00 mm/s; the compression ratio was 40%; the trigger force was 5.0 g; and the data acquisition rate was 200.00 points/s. Three parallel experiments were conducted for each group, and the results were averaged.

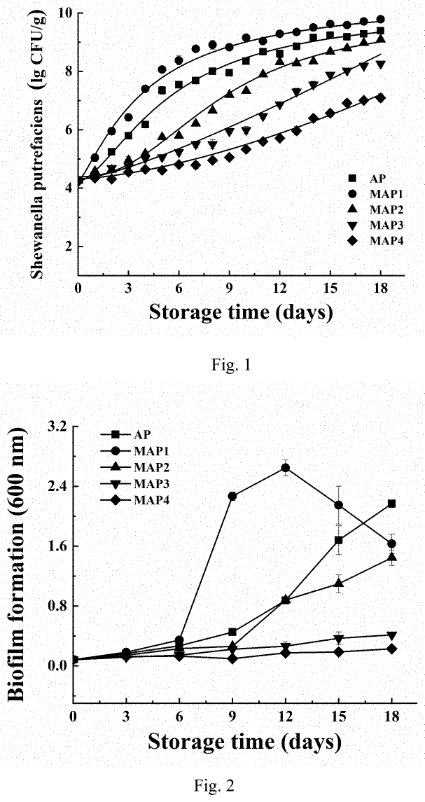

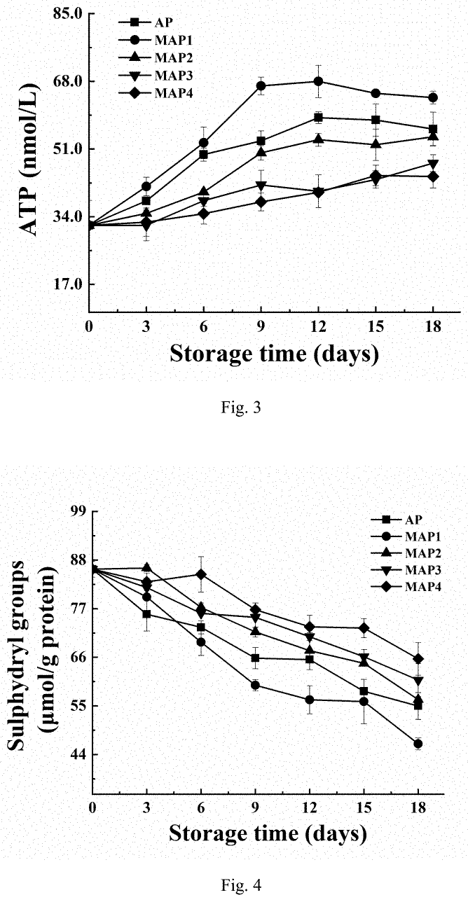

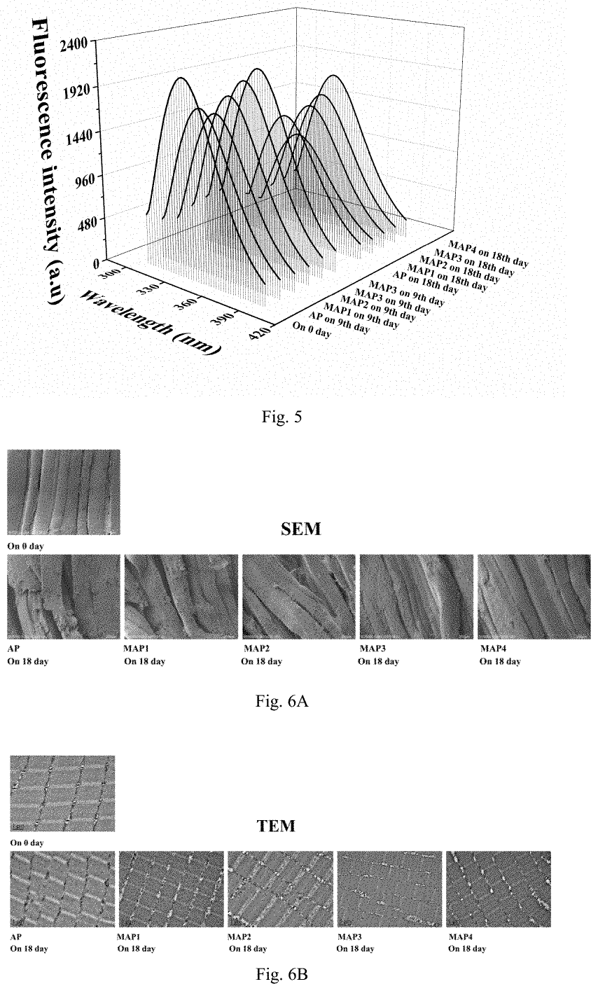

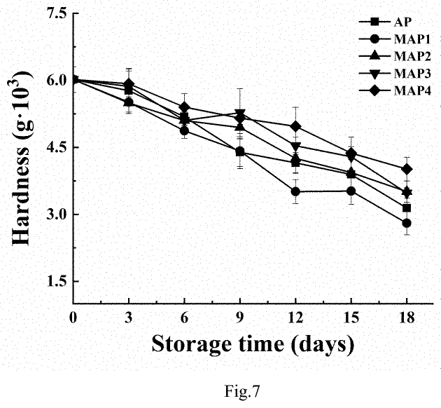

Changes of the total number of colonies, biofilm growth, adenosine triphosphate content, thiol content, myofibrillar protein tertiary structure, myofibrillar protein ultrastructure and hardness under different protective gas compositions over storage time were respectively shown in FIGS. 1-7.

It was observed that the growth and reproduction rate of the Shewanella putrefaciens stored in the presence of CO₂ was significantly lower than that of the control group, and all indicators of the large yellow croaker in the CO₂ treatment groups were superior to those of the control group, proving that the CO₂ could effectively reduce the growth and reproduction rate and the spoilage causing ability of the Shewanella putrefaciens.

### EXAMPLE 2

The method provided herein was different from that in Example 1 with respect to the following aspects.

(1) The thawed Shewanella putrefaciens was cultured in a TSB medium under shaking for 10 h to obtain a primary culture solution.

(2) The primary culture solution was inoculated into a liquid TSB medium at an inoculation amount of 0.5% by mass, and cultured under shaking for 4 h to obtain a bacterial suspension with a concentration of 10⁸ CFU/mL.

(3) An aquatic product was processed, and cut into a plurality of 80-g slices.

(4) The samples were immersed into a 70% ethanol solution for 20 s, washed with sterile distilled water 4 times, and subjected to ultraviolet sterilization for 15 min to obtain a plurality of sterilized samples, where a mass ratio of the ethanol solution to the plurality of samples was 2:1. The bacterial suspension was diluted to a bacterial concentration of 10³ CFU/mL, where the diluted bacterial suspension was 5% by weight of each sterilized sample such that a bacterial concentration of each sterilized sample was lower than or equal to 10⁵ CFU/mL.

(5) During the packaging process, a ratio of a volume of the protective gas to a weight of the sample was 2(mL):1(g), other parameters were set as follows: vacuuming time: 5 s, filling time of the protective gas: 3 s; heat sealing temperature: 150° C.; protective gas source pressure: 4 kg/cm²; and power gas source pressure: 7 kg/cm².

### EXAMPLE 3

The method provided herein was different from that in Example 1 with respect to the following aspects.

(1) The thawed Shewanella putrefaciens was cultured in a TSB medium under shaking for 14 h to obtain a primary culture solution.

(2) The primary culture solution was inoculated into a liquid TSB medium at an inoculation amount of 1.5% by mass, and cultured under shaking for 8 h to obtain a bacterial suspension with a concentration of 10⁶ CFU/mL.

(3) An aquatic product was processed, and cut into a plurality of 120-g slices.

(4) The samples were immersed into a 80% ethanol solution for 40 s, washed with sterile distilled water 2 times, and subjected to ultraviolet sterilization for 25 min to obtain a plurality of sterilized samples, where a mass ratio of the ethanol solution to the plurality of samples was 4:1. The bacterial suspension was diluted to a bacterial concentration of 10³ CFU/mL, where the diluted bacterial suspension was 15% by weight of each sterilized samples such that a bacterial concentration of each sterilized sample was lower than or equal to 10⁵ CFU/mL.

(5) During the packaging process, a ratio of a volume of the protective gas to a weight of the sample was 4(mL):1(g), other parameters were set as follows: vacuuming time: 15 s; filling time of the protective gas: 5 s; heat sealing temperature: 125° C.; protective gas source pressure: 6 kg/cm²; and power gas source pressure: 8 kg/cm².

What is claimed is:

1. A method of testing an inhibitory effect of carbon dioxide on a spoilage causing ability of Shewanella putrefaciens, comprising:
(S1) thawing frozen Shewanella putrefaciens followed by inoculation to a trypticase soy broth (TSB); and culturing the Shewanella putrefaciens under shaking for 10-14 h to obtain a primary culture solution;
(S2) inoculating the primary culture solution into a liquid medium followed by culture under shaking for 4-8 h to obtain a bacterial suspension with a concentration of 10⁶-10⁸ CFU/mL;
(S3) cutting an aquatic product material into a plurality of slices each with a mass of 80-120 g followed by washing with 0-4° C. water and drying to prepare a plurality of samples;
(S4) immersing the plurality of samples in an ethanol solution for 20-40 s followed by washing with sterile distilled water 2-4 times and ultraviolet sterilization for 15-25 min to obtain a plurality of sterilized samples; diluting the bacterial suspension to a concentration of 10³-10⁵ CFU/mL; and inoculating a diluted bacterial suspension to the plurality of sterilized samples to obtain a plurality of inoculated samples;
(S5) transferring the plurality of inoculated samples respectively to a plurality of modified atmosphere packaging bags;
(S6) turning on a modified atmosphere packaging machine and a vacuum pump to package the plurality of inoculated samples; wherein protective gases in the plurality of modified atmosphere packaging bags are each composed of 0-100% by volume of CO₂ and N₂, and vary in CO₂ content;
(S7) after packaging, storing the plurality of inoculated samples in a refrigerator at 4.0±0.5° C.; and
(S8) after stored for 0-18 days, for each protective gas, randomly selecting three inoculated samples as parallel groups for test to evaluate inhibitory effects of different concentrations of CO₂ on the spoilage causing ability of Shewanella putrefaciens.

2. The method of claim 1, wherein in step (S1), an amount of the TSB medium is 5-10 mL.

3. The method of claim 1, wherein in step (S2), an inoculation amount of the primary culture solution is 0.5-1.5% by mass.

4. The method of claim 1, wherein in step (S4), a mass concentration of the ethanol solution is 70-80%; and a mass ratio of the ethanol solution to the plurality of samples is (2-4):1.

5. The method of claim 1, wherein in step (S4), the diluted bacterial suspension is 5-15% by weight of each of the plurality of sterilized samples such that a bacterial concentration of each of the plurality of sterilized samples is lower than or equal to 10⁵ CFU/mL.

6. The method of claim 1, wherein in step (S6), a ratio of a volume the protective gas in each of the plurality of modified atmosphere packaging bags to a weight of each of the plurality of inoculated samples is 2-4 (mL):1 (g); a vacuuming time is set to 5-15 s; a filling time of the protective gas is 3-5 s; a temperature for heat sealing is 125-150° C.; a protective gas source pressure is 4-6 kg/cm²; a power gas source pressure is 7-8 kg/cm²; and after the modified atmosphere packaging machine is operated stably, a mouth of each of the plurality of modified atmosphere packaging bags containing an inoculated sample is placed at a position where gas displacement and heat sealing are performed for modified atmosphere packaging.

7. The method of claim 1, wherein in step (S8), the parallel groups are tested for colony number, biofilm growth, adenosine triphosphate content, thiol content, tertiary structure of myofibrillar protein, ultrastructure of the myofibrillar protein and hardness.
